(19) United States
(12) Patent Application Publication
(10) Pub. No.: US 2025/0268570 A1
(43) Pub. Date: Aug. 28, 2025

(54) MEDICAL INPUT APPARATUS AND ULTRASONIC IMAGING APPARATUS

(71) Applicant: GE Precision Healthcare LLC, Waukesha, WI (US)

(72) Inventors: Yalan Yang, Wuxi (CN); Kui Li, Wuxi (CN); Fenggui Huang, Wuxi (CN)

(21) Appl. No.: 19/065,887

(22) Filed: Feb. 27, 2025

(30) Foreign Application Priority Data
Feb. 28, 2024 (CN) 202410219217.5

(51) Int. Cl. A61B 8/00

(52) U.S. Cl. CPC A61B 8/4411

(57) ABSTRACT

A medical input apparatus including an input module including a body portion, a first fixing portion, and a first electrical connection portion; and an operation panel including an accommodation portion for accommodating the body portion, a second fixing portion, and a second electrical connection portion, where the input module rotates relative to the operation panel along a first rotation path and is detachably connected to the operation panel, the first fixing portion is connected to the second fixing portion when the input module is located at a first position on the first rotation path, and the first electrical connection portion is connected to the second electrical connection portion when the input module is located at a second position on the first rotation path.

### CROSS-REFERENCE TO RELATED APPLICATION

This application claim priority to Chinese Patent Application No. 202410219217.5, which was file on Feb. 28, 2024 at the Chinese Patent Office. The entire contents of the above-listed application are incorporated by reference herein in their entirety.

### TECHNICAL FIELD

Embodiments of the present application relate to the technical field of medical equipment, and in particular to a medical input apparatus and an ultrasonic imaging apparatus.

### BACKGROUND

The use of an operation panel provided with an input module in medical equipment is required, to receive control information input by a user. For example, in an ultrasonic imaging apparatus, corresponding control information is generated by detecting a direction, a speed, or a trajectory, etc. by which the user operates the input module, and then scanning imaging is performed based on the control information.

It should be noted that the above introduction of the background is only for the convenience of clearly and completely describing the technical solutions of the present application, and for the convenience of understanding for those skilled in the art.

### SUMMARY

The inventor has found that: during use of medical equipment, there is a need to detach, mount, or replace an input module. For example, detaching, mounting, or replacing of an input module is required in the case where the input module fails, the input module needs to be cleaned or maintained, the input module is not an input module desired by a user, or the like.

The input module generally is non-detachably fixed on an operation panel. Therefore, to detach, mount, or replace the input module, it is necessary to detach, mount, or replace an entire input apparatus (such as the operation panel and the input module), making the operation complex and time and labor consuming, and resulting in poor user experience. Some other prior art techniques provide a detachable input module. The detachable input module is detachably connected to the operation panel as a whole. In such a structure, it is necessary to consider a mechanical connection between the input module and the operation panel on the one hand, and to consider an electrical connection between them on the other hand. How to ensure both reliability and simplicity of the connection is still a challenge.

To resolve at least one of the above technical problems, embodiments of the present application provide a medical input apparatus and an ultrasonic imaging apparatus. It is expected to detach, mount, or replace the input module easily.

According to an aspect of the embodiments of the present application, a medical input apparatus is provided, the apparatus comprising: an input module comprising a body portion, a first fixing portion, and a first electrical connection portion; and an operation panel comprising an accommodation portion for accommodating the body portion, a second fixing portion, and a second electrical connection portion, wherein the input module rotates relative to the operation panel along a first rotation path and is detachably connected to the operation panel, the first fixing portion is connected to the second fixing portion when the input module is located at a first position on the first rotation path, and the first electrical connection portion is connected to the second electrical connection portion when the input module is located at a second position on the first rotation path.

According to another aspect of the embodiments of the present application, an ultrasonic imaging apparatus is provided, comprising the medical input apparatus as previously described.

One of the beneficial effects of the embodiments of the present application is that: the input module may be detachably connected to the operation panel through a rotation movement, and by enabling the input module to move along the rotation path, when the input module reaches a corresponding position on the rotation path, the input module may be connected to the operation panel not only mechanically but also electrically, making the operation simple and reliable, and thereby improving the user experience.

With reference to the following description and drawings, specific implementations of the embodiments of the present application are disclosed in detail, and the way in which the principles of the embodiments of the present application can be employed are illustrated. It should be understood that the embodiments of the present application are not limited in scope thereby. Within the scope of the spirit and clauses of the appended claims, the embodiments of the present application comprise many changes, modifications, and equivalents.

### BRIEF DESCRIPTION OF THE DRAWINGS

The included drawings are used to provide further understanding of the embodiments of the present application, which constitute part of the description and are used to illustrate the implementations of the present application and explain the principles of the present application together with textual description. Evidently, the drawings in the following description are merely some embodiments of the present application, and those of ordinary skill in the art may obtain other implementations according to the drawings without involving inventive effort. In the drawings:

FIG. 1 is a schematic diagram of a medical input apparatus according to an embodiment of the present application;

FIG. 2 is a schematic diagram of an input module according to an embodiment of the present application;

FIG. 3 is a schematic diagram of an operation panel according to an embodiment of the present application;

FIG. 4 is a schematic diagram of a trackball module according to an embodiment of the present application;

FIG. 5 is a schematic diagram of a trackball assembly and a first fixing portion according to an embodiment of the present application;

FIG. 6 is a schematic diagram of a second fixing portion according to an embodiment of the present application;

FIG. 7 is a schematic diagram of a touchpad module according to an embodiment of the present application;

FIG. 8 is a schematic diagram of a snap-fit state of the first fixing portion and the second fixing portion according to an embodiment of the present application;

FIG. 9 is a schematic diagram of a cover plate of the trackball module according to an embodiment of the present application;

FIG. 10 is another schematic diagram of the operation panel according to an embodiment of the present application;

FIG. 11 is another schematic diagram of the trackball module according to an embodiment of the present application;

FIG. 12 is another schematic diagram of the touchpad module according to an embodiment of the present application;

FIG. 13 is another schematic diagram of the operation panel according to an embodiment of the present application;

FIG. 14 is a schematic diagram of a retaining portion and a support portion according to an embodiment of the present application;

FIG. 15 is a schematic diagram of the support portion according to an embodiment of the present application;

FIG. 16 is a schematic diagram of the retaining portion according to an embodiment of the present application;

FIG. 17 is another schematic diagram of the retaining portion and the support portion according to an embodiment of the present application; and

FIG. 18 is a schematic diagram of an ultrasonic imaging apparatus according to an embodiment of the present application.

### DETAILED DESCRIPTION

The foregoing and other features of the embodiments of the present application will become apparent from the following description with reference to the drawings. In the description and drawings, specific implementations of the present application are disclosed in detail, and part of the implementations in which the principles of the embodiments of the present application may be employed are indicated. It should be understood that the present application is not limited to the described implementations. On the contrary, the embodiments of the present application include all modifications, variations, and equivalents which fall within the scope of the appended claims.

In the embodiments of the present application, the terms “first”, “second”, etc., are used to distinguish different elements, but do not represent a spatial arrangement or temporal order, etc., of these elements, and these elements should not be limited by these terms. The term “and/or” includes any and all combinations of one or more associated listed terms. The terms “comprise”, “include”, “have”, etc., refer to the presence of described features, elements, components, or assemblies, but do not exclude the presence or addition of one or more other features, elements, components, or assemblies.

In the embodiments of the present application, the singular forms “a”, “the”, etc., include plural forms, and should be broadly construed as “a type of” or “a class of” rather than being limited to the meaning of “one”. Furthermore, the term “the” should be construed as including both the singular and plural forms, unless otherwise specified in the context. In addition, the term “according to” should be construed as “at least in part according to . . . ” and the term “on the basis of” should be construed as “at least in part based on . . . ”, unless otherwise specified in the context.

In the description of the present application, it should be noted that unless otherwise explicitly specified and defined, the terms “connecting” and “connection” should be understood in a broad sense, for example, they may be a fixed connection, a detachable connection, or an integrated connection; may be a mechanical connection or an electrical connection; and may be a direct connection, or an indirect connection by means of an intermediate medium. Those of ordinary skill in the art can understand the specific meaning of the aforementioned terms in the present application according to the specific situation.

The features described and/or illustrated for one implementation may be used in one or more other implementations in the same or similar way, be combined with features in other embodiments, or replace features in other implementations. The terms “include/comprise” when used herein refer to the presence of features, integrated components, steps, or assemblies, but do not preclude the presence or addition of one or more other features, integrated components, steps, or assemblies.

In addition, in the following description of the present application, for the convenience of description, a direction perpendicular to the operation panel is referred to as an “axial direction” (an up-down direction or a vertical direction), a radius direction using the axial direction as the center is referred to as a “radial direction” (a horizontal direction), and a direction around the axial direction is referred to as a “circumferential direction”. However, it should be noted that these are just for the convenience of description, and do not limit an orientation of the medical input apparatus during manufacturing and use.

The embodiments of the present application are specifically described below.

The embodiments of the present application provide a medical input apparatus.

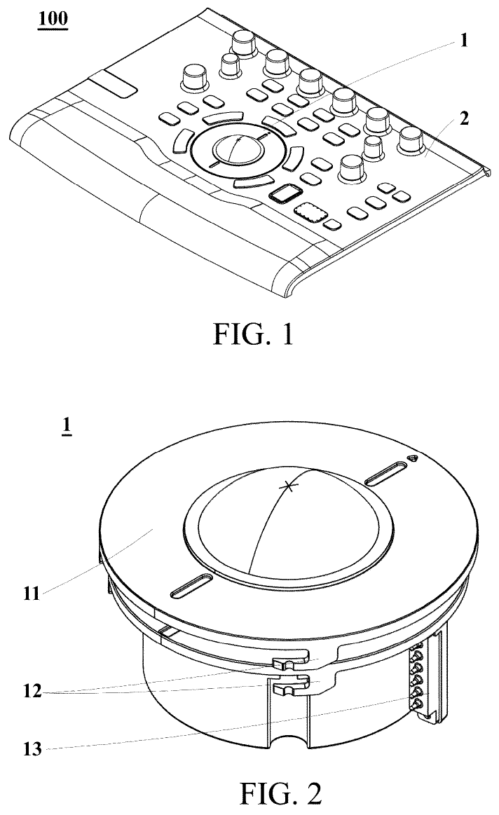

FIG. 1 is a schematic diagram of a medical input apparatus according to an embodiment of the present application. As shown in FIG. 1, the medical input apparatus 100 includes: an input module 1 and an operation panel 2.

FIG. 2 is a schematic diagram of an input module according to an embodiment of the present application. As shown in FIG. 2, the input module 1 includes a body portion 11, a first fixing portion 12, and a first electrical connection portion 13.

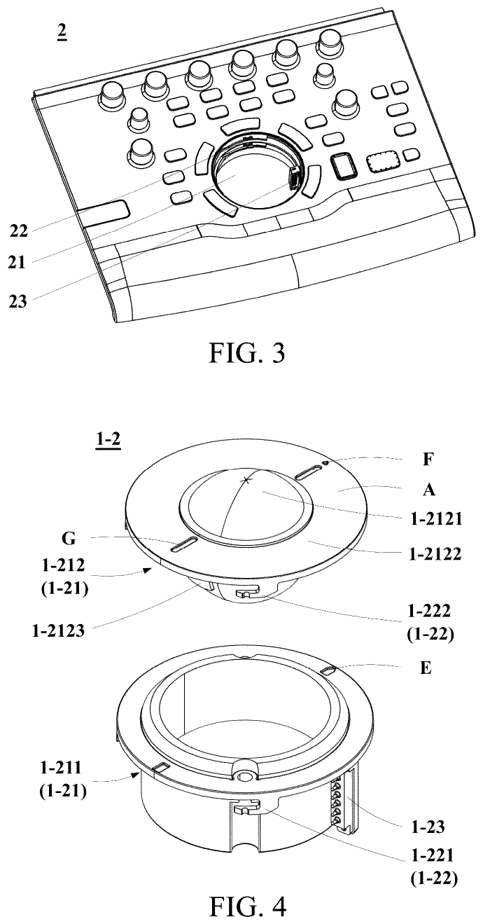

FIG. 3 is a schematic diagram of an operation panel according to an embodiment of the present application. As shown in FIG. 3, the operation panel 2 includes an accommodation portion 21 for accommodating the body portion 11, a second fixing portion 22, and a second electrical connection portion 23.

The input module 1 rotates relative to the operation panel 2 along a first rotation path and is detachably connected to the operation panel 2, the first fixing portion 12 is connected to the second fixing portion 22 when the input module 1 is located at a first position on the first rotation path, and the first electrical connection portion 13 is connected to the second electrical connection portion 23 when the input module 1 is located at a second position on the first rotation path.

According to the above embodiment, in the medical input apparatus 100, the input module 1 is detachably connected to the operation panel 2, so as to facilitate a user in mounting, detaching, or replacing the input module 1 as needed. In addition, the input module may be detachably connected to the operation panel 2 through a rotation movement, and by enabling the input module 1 to move along the rotation path, when the input module 1 reaches a corresponding position on the rotation path, the input module 1 may be connected to the operation panel 2 not only mechanically but also electrically, making the operation simple and reliable.

In some embodiments, FIG. 1 to FIG. 3 illustrate the medical input apparatus with an example in which the input module 1 is a trackball module. The present application is not limited thereto, and the input module 1 may alternatively be a touchpad module. Alternatively, the input module 1 may include a trackball module and a touchpad module, the trackball module and the touchpad module being alternately connected to the operation panel 2.

Exemplary illustration of the input module is provided below by using the trackball module and the touchpad module as examples, respectively.

FIG. 4 is a schematic diagram of the trackball module according to an embodiment of the present application. As shown in FIG. 4, a body portion 1-21 of the trackball module 1-2 includes a base assembly 1-211 and a trackball assembly 1-212, the base assembly 1-211 being detachably assembled with the trackball assembly 1-212. The base assembly 1-211 is enabled to be detachably assembled with the trackball assembly 1-212, so as to facilitate cleaning or replacing of the base assembly 1-211 and the trackball assembly 1-212.

In some embodiments, as shown in FIG. 4, the first electrical connection portion 1-23 may be provided on the base assembly 1-211. The present application is not limited thereto, and the first electrical connection portion 1-23 may alternatively be provided on the trackball assembly 1-212.

In some embodiments, the first fixing portion 1-22 may be provided on the base assembly 1-211 and the trackball assembly 1-212. The present application is not limited thereto, and the first fixing portion 1-22 may alternatively be provided on one of the base assembly 1-211 and the trackball assembly 1-212.

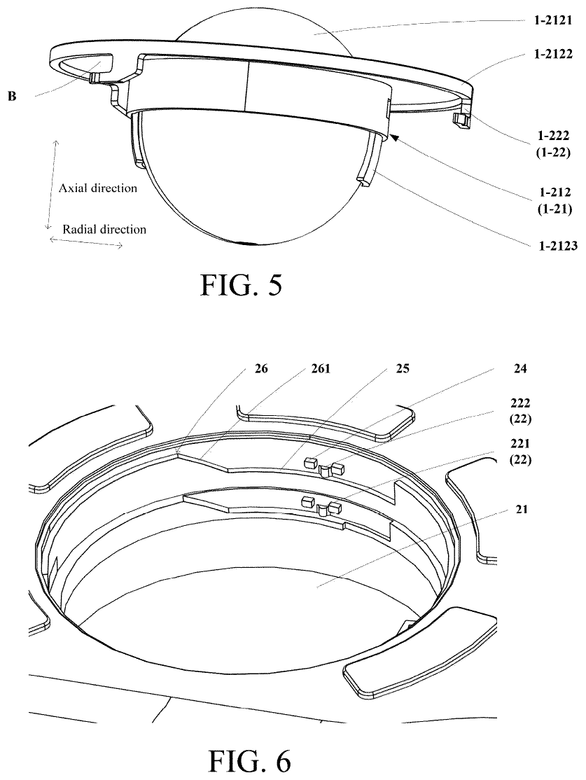

FIG. 5 is a schematic diagram of the trackball assembly and the first fixing portion according to an embodiment of the present application. In some embodiments, as shown in FIGS. 4 and 5, the trackball assembly 1-212 may include a ball 1-2121 and a cover plate 1-2122. The cover plate 1-2122 includes a first surface A (shown in FIG. 4) and a second surface B (shown in FIG. 5), an opening portion passing through the first surface A and the second surface B is provided at the center of the cover plate 1-2122, and a claw portion 1-2123 is provided at a side of the second surface B. Part of the ball 1-2121 is exposed from the opening portion, and the ball is retained by the claw portion 1-2123 in a freely rotatable state.

In some embodiments, there may be one or more claw portions 1-2123.

For example, as shown in FIG. 5, there are a plurality of claw portions 1-2123. An end of the claw portion 1-2123 is connected to the second surface (which may be a direct connection or a connection by means of another component), and the claw portion 1-2123 includes a portion extending axially and radially inward relative to the end, whereby the claw portion 1-2123 can support the ball 1-2121 in the axial direction.

For another example, there may be one claw portion 1-2123. An end of the claw portion is connected to the second surface (which may be a direct connection or a connection by means of another component), and the other end thereof is provided with a bearing portion. The bearing portion supports the ball 1-2121 in the axial direction. The bearing portion may be various structures. For example, the bearing portion may be a circular ring having an opening. By providing the opening, the ball 1-2121 may be easily detached from or assembled with the cover plate 1-2122, thereby facilitating cleaning, replacing, etc. of the ball 1-2121 and the cover plate 1-2122.

In some embodiments, the base assembly 1-211 may be detachably assembled with the trackball assembly 1-212 in various manners.

For example, the base assembly 1-211 and the trackball assembly 1-212 are each detachably connected to the operation panel 2. As shown in FIG. 4, the first fixing portion 1-22 includes a fifth fixing portion 1-221 provided on the base assembly 1-211 and a sixth fixing portion 1-222 provided on the trackball assembly 1-212.

FIG. 6 is a schematic diagram of the second fixing portion according to an embodiment of the present application. As shown in FIG. 6, corresponding to the fifth fixing portion 1-221 and the sixth fixing portion 1-222 of the first fixing portion 1-22, the second fixing portion 22 may include a seventh fixing portion 221 and an eighth fixing portion 222 that are provided in the accommodation portion 21.

By connecting the fifth fixing portion 1-221 to the seventh fixing portion 221 and the sixth fixing portion 1-222 to the eighth fixing portion 222, the trackball assembly 1-212 and the base assembly 1-211 can be assembled together, and the trackball module 1-2 can be mounted to the operation panel 2.

The present application is not limited thereto, and the base assembly 1-211 may be detachably assembled with the trackball assembly 1-212 in another manner. For example, the base assembly 1-211 may include a ninth fixing portion, and the trackball assembly 1-212 may include a tenth fixing portion, where the ninth fixing portion is detachably connected to the tenth fixing portion, thereby enabling the base assembly 1-211 to be detachably assembled with the trackball assembly 1-212. In this case, the first fixing portion 1-22 may be provided on the base assembly 1-211, on the trackball assembly 1-212, or on the base assembly 1-211 and the trackball assembly 1-212.

In some embodiments, in a state where the trackball module 1-2 is mounted to the operation panel 2, the ball 1-2121 in the trackball assembly 1-212 may abut a component of the base assembly 1-211. Control information is generated by detecting a path and/or speed of the ball 1-2121, and is transmitted by the first electrical connection portion 1-23 to the second electrical connection portion 23 of the operation panel 2, so as control other components of the ultrasound imaging apparatus to perform related actions.

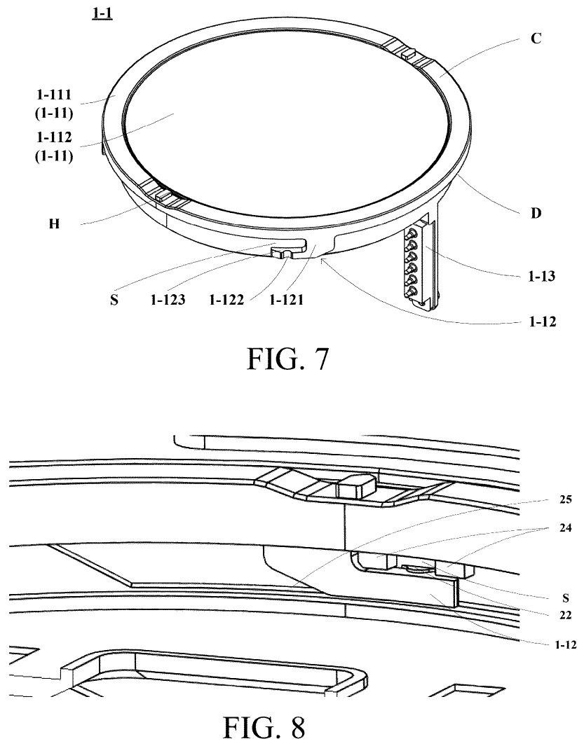

FIG. 7 is a schematic diagram of the touchpad module according to an embodiment of the present application. As shown in FIG. 7, in the touchpad module 1-1, a first fixing portion 1-12 and a first electrical connection portion 1-13 are provided on a body portion 1-11.

For example, the body portion 1-11 includes a cover plate 1-111 and a touchpad 1-112, where the touchpad 1-112 is provided at an inner side of the cover plate 1-111 in the circumferential direction. The cover plate 1-111 includes a first face C and a second face D, where the first fixing portion 1-12 and the first electrical connection portion 1-13 are provided at a side of the second face D.

The first electrical connection portion 1-13 is electrically connected to the touchpad 1-112. Control information is generated by detecting a path and/or speed of a user finger etc. on the touchpad 1-112, and is transmitted by the first electrical connection portion 1-13 to the second electrical connection portion 23 of the operation panel 2, so as control other components of the ultrasound imaging apparatus to perform related actions.

In some embodiments, the first fixing portion may be connected to the second fixing portion in various manners. For example, the connection may be a snap-fit connection, a threaded connection, etc.

Exemplary illustration of the connection manner between the first fixing portion and the second fixing portion is provided below by using the first fixing portion 1-12 of the touchpad module 1-1 and the second fixing portion 22 of the operation panel 2 as examples. It may be understood that the following illustration of the connection manner between the first fixing portion and the second fixing portion is also applicable to the first fixing portion 1-22 of the trackball module 1-2 (for example, the fifth fixing portion 1-221 and the sixth fixing portion 1-222) and the second fixing portion 22 of the operation panel 2 (the seventh fixing portion 221 and the eighth fixing portion 222).

Taking the snap-fit connection as an example, as shown in FIG. 7, the first fixing portion 1-12 may have a plate-shaped portion 1-121 extending from the second face D in the axial direction, and a groove 1-122 extending in the axial direction is provided at a side of the plate-shaped portion 1-121 in the circumferential direction.

As shown in FIG. 6, the second fixing portion 22 may be a protruding portion that protrudes inward from a side wall of the accommodation portion 21 in the radial direction. Through snap-fit between the groove 1-122 and the protruding portion, the first fixing portion 1-12 can be connected to the second fixing portion 22, and mounting or detaching of the input apparatus can be facilitated.

The present application is not limited thereto, and the first fixing portion 1-12 may alternatively be other structures. For example, in the first fixing portion 1-12, a protruding portion is provided at a side of the plate-shaped portion 1-121 in the circumferential direction. The second fixing portion may have a groove that is depressed outward from the side wall of the accommodation portion 21 in the radial direction, and the protruding portion is snap-fitted with the groove.

The first fixing portion 1-12 may be made of a flexible material, so as to facilitate the snap-fit.

In some embodiments, the groove 1-122 and the second face D may be in no contact. For example, as shown in FIG. 7, the groove 1-122 is provided on an extension portion 1-123 of the plate-shaped portion 1-121 that extends in the circumferential direction, where the groove 1-122 is spaced apart from the second face D by a gap S therebetween. As such, the elasticity of the first fixing portion 1-12 can be improved, and the touchpad module 1-1 can be guided via the gap S. In addition, snap-fit can be achieved via the gap S between the groove and the second face D. Therefore, the reliability of snap-fit can be further improved, and mounting or detaching of the trackball apparatus can be facilitated.

FIG. 8 is a schematic diagram of a snap-fit state of the first fixing portion and the second fixing portion according to an embodiment of the present application. As shown in FIGS. 6 and 8, the operation panel 2 may further include: a position-limiting portion 24. The position-limiting portion 24 may be located at an upper side of the second fixing portion 22.

As shown in FIG. 8, in a state where the first fixing portion 1-12 is snap-fitted with the second fixing portion 22, the position-limiting portion 24 is accommodated in the gap S. The first fixing portion 1-12 may abut against the position-limiting portion 24 in the up-down direction. As such, the input module 1 can be limited in the up-down direction.

In some embodiments, as shown in FIGS. 6 and 8, the operation panel 2 may further include: a step portion 25 extending in the circumferential direction, where the step portion 25 may be located at a lower side of the second fixing portion.

As shown in FIG. 8, in a state where the first fixing portion 1-12 is snap-fitted with the second fixing portion 22, the step portion 25 is located at a lower side of first fixing portion 1-12. The first fixing portion 1-12 may abut against the step portion 25 in the up-down direction. As such, the input module 1 can be limited in the up-down direction.

In some embodiments, in the trackball module 1-2, the fifth fixing portion 1-221 and the sixth fixing portion 1-222 of the first fixing portion 1-22 may be a structure the same as or similar to the first fixing portion 1-12 of the touchpad module 1-1. As such, the trackball module 1-2 and the touchpad module 1-1 can be mounted on one operation panel 2 in a compatible manner.

In some embodiments, as shown in FIG. 6, the second fixing portion 22 of the operation panel 2 includes the seventh fixing portion 221 and the eighth fixing portion 222, where the seventh fixing portion 221 and the eighth fixing portion 222 are connected to the fifth fixing portion 1-221 and the sixth fixing portion 1-222 of the first fixing portion 1-22 of the trackball module 1-2, respectively. When the touchpad module 1-1 is mounted in the operation panel 2, the first fixing portion 1-12 of the touchpad module 1-1 may be connected to the seventh fixing portion 221. The present application is not limited thereto, and the first fixing portion 1-12 of the touchpad module 1-1 may alternatively be connected to the eighth fixing portion 222.

In some embodiments, there may be a plurality of first fixing portions 12, and correspondingly, there may be a plurality of second fixing portions 22, where the plurality of first fixing portions 12 and the plurality of second fixing portions 22 may be provided non-uniformly in the circumferential direction of the body portion. As such, the user may easily confirm an orientation of the input module 1, thereby facilitating mounting the input module 1 in the accommodation portion 21 of the operation panel 2 in the correct orientation.

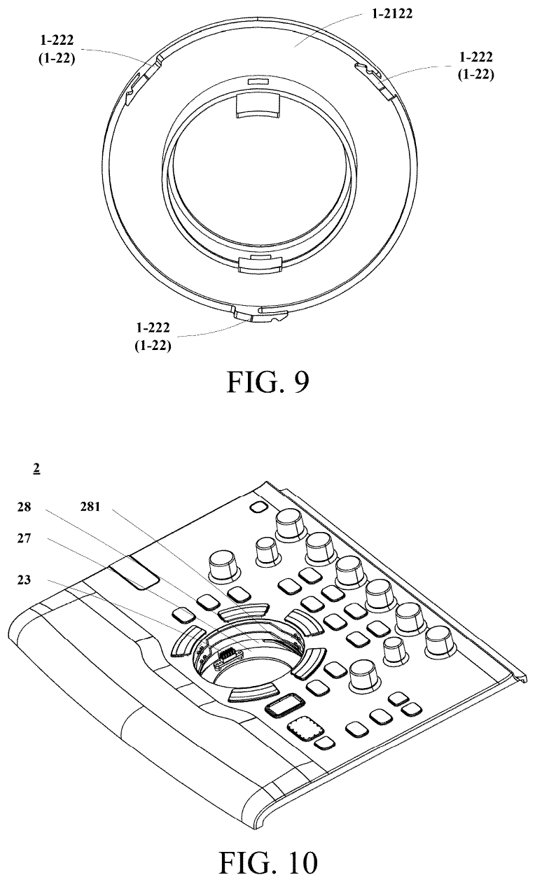

FIG. 9 is a schematic diagram of the cover plate of the trackball module according to an embodiment of the present application. As shown in FIG. 9, three first fixing portions 1-22 are provided on the cover plate 1-2122, where there are different spacings between the three first fixing portions 1-22 in the circumferential direction. The present application is not limited thereto, and the first fixing portions and the second fixing portions may alternatively be provided uniformly in the circumferential direction of the body portion.

In some embodiments, the input module 1 may be provided with a mark used for marking the orientation of the input module 1. As such, the user can confirm the orientation of the input module 1 according to the mark. For example, as shown in FIG. 4, in the trackball module 1-2, marks E and F may be provided in the base assembly 1-211 and the trackball assembly 1-212, respectively.

In some embodiments, the input module 1 may perform a rotational downward movement or a rotational upward movement along at least part of the first rotation path.

For example, a process of the input module 1 moving from a start point of the first rotation path to an end point of the first rotation path is a process of mounting the input module 1 to the operation panel 2. A process of the input module 1 moving from the end point of the first rotation path to the start point of the first rotation path is a process of detaching the input module 1 from the operation panel 2.

When the input module 1 is located at the start point of the first rotation path, an upper end face of the input module 1 may be located at a position higher than an upper end face of the operation panel 2. As such, the user can easily remove the input module 1 from the operation panel 2.

When the input module 1 is located at the end point of the first rotation path, the upper end face of the input module 1 may be flush with the upper end face of the operation panel 2. As such, the user can easily operate the input module 1.

In some embodiments, the input module 1 may be provided with a force applying portion, so that the user can apply a rotational force to the input module 1 via the force applying portion, facilitating detaching and mounting operations for the input module 1. For example, as shown in FIG. 4, in the trackball module 1-2, a force applying portion G may be provided in the trackball assembly 1-212. As shown in FIG. 7, a force applying portion H may be provided in the touchpad module 1-1.

In some embodiments, the rotation of the input module 1 along the first rotation path may be enabled in various manners. For example, as shown in FIG. 6, the operation panel 2 may be provided with a first step portion 26. The first step portion 26 protrudes inward in the radial direction from the side wall of the accommodation portion 21 of the operation panel 2. The first step portion 26 includes at least an inclined portion 261, where the inclined portion 261 extends in the circumferential direction and the axial direction.

At least part of a lower surface of the first fixing portion 11 of the input module 1 abuts against an upper surface of the first step portion 26. In this state, the input module 1 moves along the first step portion 26, and can perform a rotational downward movement or a rotational upward movement when passing through the inclined portion 261.

In some embodiments, an end of the first step portion 26 may be connected to and flush with the step portion 25 mentioned above. As such, the input module 1 may be enabled to reliably rotate to a position connected to the second fixing portion 22.

In some embodiments, the other end of the first step portion 26 (corresponding to the start point of the first rotation path) may be horizontal, whereby the input module 1 can be stably placed on the other end of the first step portion 26, preventing an undesired movement of the input module 1, and thereby facilitating a user operation.

The present application is not limited thereto, and the rotation of the input module 1 along the first rotation path may be enabled in other manners. For example, a grooved guide rail etc. is provided on the side wall of the accommodation portion 21 of the operation panel 2.

In some embodiments, the first fixing portion 12 of the input module 1 may be connected to the second fixing portion 22 of the operation panel 2 when the input module 1 is located at the first position on the first rotation path. The first electrical connection portion 13 of the input module 1 may be connected to the second electrical connection portion 23 of the operation panel 2 when the input module 1 is located at the second position on the first rotation path. The first position and the second position may be various positions on the first rotation path.

For example, the first position and the second position may refer to an end point of the first rotation path. In other words, when the input module 1 rotates to the end point along the first rotation path, not only a mechanical connection between the first fixing portion 12 and the second fixing portion 22 but also an electrical connection between the first electrical connection portion 13 and the second electrical connection portion 23 can be achieved. As such, in order to mechanically and electrically connect the input module 1 to the operation panel 2, the user just needs to rotate the input module 1 to the end point of the first rotation path, without an operation of plugging or unplugging the first electrical connection portion 13 and the second electrical connection portion 23, making the operation easy and quick.

In some embodiments, the first electrical connection portion 13 may be connected to the second electrical connection portion 23 in a non-pluggable manner. For example, when the input module 1 is located at the end point of the first rotation path, the first electrical connection portion 13 and the second electrical connection portion 23 abut against each other.

As shown in FIGS. 2 and 3, the first electrical connection portion 13 and the second electrical connection portion 23 may be spring-loaded pin interfaces (such as pogo-pin interfaces). As such, the reliability of the electrical connection can be improved.

In some embodiments, an abutting direction of the first electrical connection portion 13 and the second electrical connection portion 23 may be the same as a connection direction of the first fixing portion 12 and the second fixing portion 22. For example, as shown in FIG. 8, the first fixing portion 1-12 is snap-fitted with the second fixing portion 22 in the horizontal direction, and the first electrical connection portion 13 abuts against the second electrical connection portion 23 in the horizontal direction.

In some embodiments, the first electrical connection portion 13 may be provided at a position offset from the first fixing portion 12 in the circumferential direction, thereby avoiding interference between them. Similarly, the second electrical connection portion 23 may be provided at a position offset from the second fixing portion 22 in the circumferential direction.

Exemplary illustration of steps of replacing the input module 1 is provided below using an example in which the touchpad module is replaced with the trackball module and the first electrical connection portion is connected to the second electrical connection portion in the non-pluggable manner. It should be understood that the following illustration is also applicable to a case where the trackball module is replaced with the touchpad module, except that the following steps are performed in a reverse order.

Step 1: The touchpad module 1-1 is enabled to rotate along a first rotation path-1 from an end point of the first rotation path-1 to a start point of the first rotation path-1. At this time, the first fixing portion 1-12 of the touchpad module 1-1 is released from the mechanical connection with the second fixing portion 22 of the operation panel 2; the first electrical connection portion 1-13 of the touchpad module 1-1 is electrically disconnected from the second electrical connection portion 23 of the operation panel 2; and an upper end face of the touchpad module 1-1 is located at a position higher than the upper end face of the operation panel 2, thereby facilitating the user in taking out the touchpad module 1-1 from the accommodation portion 21 of the operation panel 2.

Step 2: The base assembly 1-211 of the trackball module 1-2 is enabled to rotate along the first rotation path-1 from the start point of the first rotation path-1 to the end point of the first rotation path-1. At this time, the fifth fixing portion 1-221 of the base assembly 1-211 is mechanically connected to the seventh fixing portion 221 of the operation panel 2; and the first electrical connection portion 1-23 of the trackball module 1-2 is electrically connected to the second electrical connection portion 23 of the operation panel 2.

Step 3: The trackball assembly 1-212 of the trackball module 1-2 is enabled to rotate along a first rotation path-2 from a start point of the first rotation path-2 to an end point of the first rotation path-2 (where the first rotation path includes the first rotation path-1 and the first rotation path-2. The first rotation path-2 is parallel to the first rotation path-1, and the first rotation path-2 is located at an upper side of the first rotation path-1). At this time, the sixth fixing portion 1-222 of the trackball assembly 1-212 is mechanically connected to the eighth fixing portion 222 of the operation panel 2. As such, the operation of replacing the touchpad module with the trackball module 1-2 is completed.

In some embodiments, the first position and the second position may alternatively be other positions on the first rotation path. For example, the first position may be the end point of the first rotation path, and the second position may be the start point of the first rotation path. In other words, when the input module 1 is at the start point of the first rotation path, the first electrical connection portion 13 is connected to the second electrical connection portion 23, and the input module 1 rotates along the first rotation path in a state where the connection between the first electrical connection portion 13 and the second electrical connection portion 23 is maintained. When the input module 1 rotates to the end point of the first rotation path, the first fixing portion 12 is connected to the second fixing portion 22. As such, in order to electrically and mechanically connect the input module 1 to the operation panel 2, the user just needs to place the input module 1 at the start point of the first rotation path and then rotate it to the end point of the first rotation path, thereby facilitating the operation and ensuring the reliability of the electrical connection.

In some embodiments, the rotation of the second electrical connection portion 23 with the input module 1 to the second position may be enabled in various manners.

FIG. 10 is another schematic diagram of the operation panel according to an embodiment of the present application. As shown in FIG. 10, the operation panel 2 may further include: a retaining portion 27. The retaining portion 27 is fixedly connected to the second electrical connection portion 23, and rotates along a second rotation path parallel to the first rotation path.

For example, in a state where the first electrical connection portion 13 is connected to the second electrical connection portion 23, the retaining portion 27 rotates in synchronization with the input module 1.

In some embodiments, the first electrical connection portion 13 may be connected to the second electrical connection portion 23 in a pluggable manner. As such, the input module 1 can reliably drive the retaining portion 27 to rotate in synchronization with it.

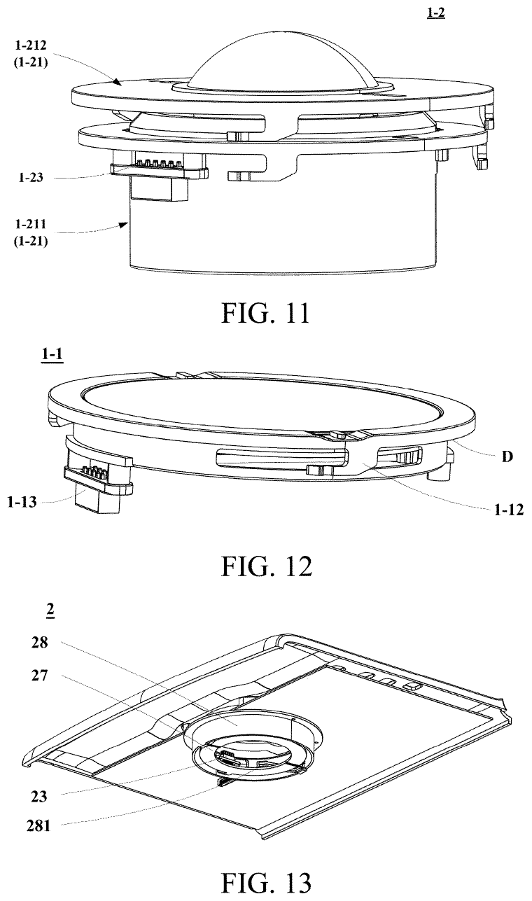

FIG. 11 is another schematic diagram of the trackball module according to an embodiment of the present application. As shown in FIG. 11, the first electrical connection portion 1-23 may be provided on the base assembly 1-211. As shown in FIGS. 10 and 11, the first electrical connection portion 1-23 and the second electrical connection portion 23 can be connected by plugging in the axial direction.

FIG. 12 is another schematic diagram of the touchpad module according to an embodiment of the present application. As shown in FIG. 12, the first electrical connection portion 1-13 may be provided at a side of the second face D of the touchpad module 1-1. As shown in FIGS. 10 and 12, the first electrical connection portion 1-13 and the second electrical connection portion 23 can be connected by plugging in the axial direction.

In some embodiments, when the input module 1 is located at the start point of the first rotation path and the retaining portion 27 is located at the first end of the second rotation path, the first electrical connection portion 13 and the second electrical connection portion 23 may overlap each other (e.g., overlap each other in the axial direction). In other words, when the input module 1 is placed at the start point of the first rotation path and the retaining portion 27 is placed at the first end of the second rotation path, the first electrical connection portion 13 may be connected to the second electrical connection portion 23. For example, the second electrical connection portion 23 may be plugged into the first electrical connection portion 13, or the first electrical connection portion 13 may be plugged into the second electrical connection portion 23.

In some embodiments, the first electrical connection portion 13 and the second electrical connection portion 23 may be plug-in interfaces. For example, they can be coupled in a pluggable manner. As such, the reliability and service life of the electrical connection can be improved.

In some embodiments, the rotation of the retaining portion 27 along the second rotation path may be enabled in various manners. FIG. 13 is another schematic diagram of the operation panel according to an embodiment of the present application. As shown in FIGS. 10 and 13, the operation panel 2 may further include: a support portion 28. The support portion 28 includes a first guide member 281. The retaining portion 27 may include a second guide member. In a state where the first guide member 281 is engaged with the second guide member, the retaining portion 27 rotates along the second rotation path.

In some embodiments, as shown in FIG. 13, the support portion 28 may be provided at a lower side of the accommodation portion 21. The present application is not limited thereto, and the support portion 28 may alternatively be provided in the accommodation portion 21, etc.

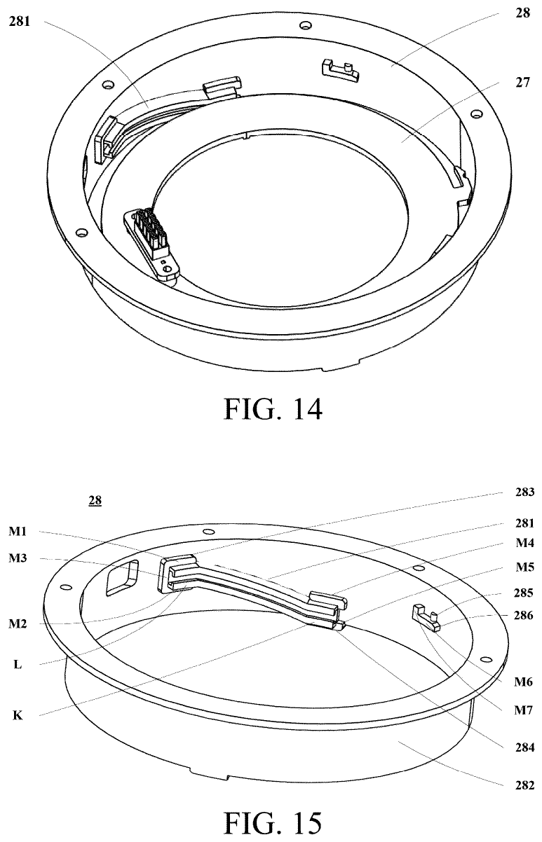

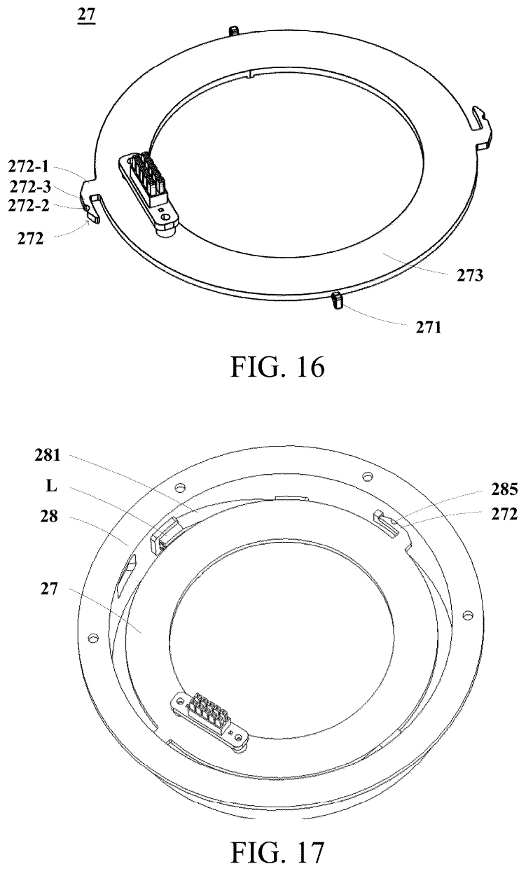

In some embodiments, the second guide member of the retaining portion 27 and the first guide member of the support portion 28 may be of various forms. FIG. 14 is a schematic diagram of the retaining portion and the support portion according to an embodiment of the present application. FIG. 15 is a schematic diagram of the support portion according to an embodiment of the present application. FIG. 16 is a schematic diagram of the retaining portion according to an embodiment of the present application.

As shown in FIGS. 14 to 16, the first guide member 281 of the support portion 28 may be a guide rail (shown in FIGS. 14 and 15), and the second guide member 271 of the retaining portion 27 (shown in FIG. 16) may be a protrusion extending outward from a body portion 273 of the retaining portion 27 in the radial direction. By engaging the protrusion in the guide rail, the retaining portion 27 may be enabled to move along the second rotation path. The present application is not limited thereto, and the first guide member 281 and the second guide member 271 may alternatively be in other forms. For example, the first guide member 281 is a protrusion, and the second guide member 271 is a guide rail, a step portion, etc.

In some embodiments, as shown in FIG. 15, the support portion 28 may include a cylindrical portion 282, and the first guide member 281 may be provided on a side wall of the cylindrical portion 282. The first guide member 281 may be connected to the cylindrical portion 282 in various manners. As shown in FIG. 15, the first guide member 281 as being a guide rail is used as an example, and the support portion 28 may further include an eleventh fixing portion 283 and a twelfth fixing portion 284, where the eleventh fixing portion 283 and the twelfth fixing portion 284 are fixed on the side wall of the cylindrical portion 282. The eleventh fixing portion 283 abuts against a first end L of the first guide member 281; and the twelfth fixing portion 284 abuts against a second end K of the first guide member 281. As such, the first guide member 281 can be mounted to the cylindrical portion 282. The present application is not limited thereto, and the first guide member 281 and the cylindrical portion 282 may alternatively be connected by means of welding, adhesion, integral molding, etc.

In some embodiments, the first end L of the first guide member 281 may correspond to the first end of the second rotation path, and the second end K of the first guide member 281 may correspond to the second end of the second rotation path. That is, when the second guide member 271 of the retaining portion 27 is located at the first end L of the first guide member 281 of the support portion 28, the retaining portion 27 is located at the first end of the second rotation path; and when the second guide member 271 of the retaining portion 27 is located at the second end K of the first guide member 281 of the support portion 28, the retaining portion 27 is located at the second end of the second rotation path.

As shown in FIG. 15, the eleventh fixing portion 283 includes a first portion M1 and a second portion M2 that extend in the circumferential direction, and a third portion M3 extending in the axial direction, where the first portion M1 and the second portion M2 each limit the first guide member 281 in the axial direction, and the third portion M3 limits the first guide member 281 in the circumferential direction.

As shown in FIG. 15, the third portion M3 may be provided as a structure overlapping at least part of a groove of the first guide member 281 in the circumferential direction. As such, when the second guide member 271 of the retaining portion 27 is located at the first end L of the first guide member 281, the third component can prevent the second guide member 271 of the retaining portion 27 from sliding out of the groove of the first guide member 281.

As shown in FIG. 15, the twelfth fixing portion 284 includes a fourth portion M4 and a fifth portion M5 that extend in the circumferential direction, where the fourth portion M4 and the fifth portion M5 each limit the first guide member 281 in the axial direction.

The twelfth fixing portion 284 may keep the second end K of the first guide member 281 in an open state, so that the second guide member 271 can slide into the groove of the first guide member 281 from the second end of the first guide member 281, thereby facilitating detaching or assembly of the retaining portion 27 and the support portion 28.

The present application is not limited thereto, and the first end L and the second end K of the first guide member 281 may both be in an open state; the first end L and the second end K of the first guide member 281 may both be in a closed state; or the first end L of the first guide member 281 is in an open state, and the second end K thereof is in a closed state.

FIG. 17 is another schematic diagram of the retaining portion and the support portion according to an embodiment of the present application. FIG. 17 illustrates a state where the retaining portion 27 is located at the first end of the second rotation path. In some embodiments, as shown in FIGS. 15 to 17, the support portion 28 may further include a third fixing portion 285 (shown in FIGS. 15 and 17) and the retaining portion 27 includes a fourth fixing portion 272 (shown in FIGS. 16 and 17).

As shown in FIG. 17, when the retaining portion 27 is located at the first end of the second rotation path (for example, the second guide member 271 of the retaining portion 27 is located at the first end L of the first guide member 281 of the support portion 28), the third fixing portion 285 is connected to the fourth fixing portion 272. As such, the retaining portion 27 can be accurately positioned at the first end of the second rotation path.

In some embodiments, the third fixing portion 285 and the fourth fixing portion 272 may be connected in various manners. For example, as shown in FIG. 17, the third fixing portion 285 and the fourth fixing portion 272 are connected by snap fit.

As shown in FIG. 15, the third fixing portion 285 may be a protruding portion that protrudes inward from the side wall of the cylindrical portion 282 of the support portion 28 in the radial direction. As shown in FIG. 16, the fourth fixing portion 272 may have a plate-shaped portion 272-1 extending outward from the body portion 273 in the radial direction, and a groove 272-2 extending in the axial direction may be provided at a side of the plate-shaped portion 1-121 in the circumferential direction. The third fixing portion 285 may be connected to the fourth fixing portion 272 by means of snap-fit between the groove 272-2 and the protruding portion. The present application is not limited thereto, and the first fixing portion 1-12 may alternatively be other structures.

The fourth fixing portion 272 may be made of a flexible material, so as to facilitate the snap-fit.

In some embodiments, for example, as shown in FIG. 16, the groove 272-2 may be provided on an extension portion 272-3 of the plate-shaped portion 272-1 that extends in the circumferential direction, where the extension portion 272-3 is spaced apart from the body portion 273 by a gap therebetween. As such, the elasticity of the first fixing portion 1-12 can be improved, thereby facilitating the mounting or detaching.

In some embodiments, as shown in FIG. 15, the support portion 28 may further include a third position-limiting portion 286. The third position-limiting portion 286 includes a sixth portion M6, where the sixth portion M6 is located at a lower side of the third fixing portion 285, and when the retaining portion 27 is located at the first end of the second rotation path, the sixth portion M6 can support the fourth fixing portion 272 of the retaining portion 27 upward. Since the first electrical connection portion 13 of the input module 1 is connected to the second electrical connection portion 23 on the retaining portion 27 when the retaining portion 27 is located at the first end of the second rotation path, the retaining portion 27 can be reliably supported by providing the sixth portion M6.

In some embodiments, as shown in FIG. 15, the third position-limiting portion 286 may further include a seventh portion M7, where the seventh portion M7 is located at a side of the third fixing portion 285 in the circumferential direction, and when the retaining portion 27 is located at the first end of the second rotation path, the seventh portion M7 can abut against a circumferential end portion of the fourth fixing portion 272 of the retaining portion 27 in the circumferential direction. By providing the seventh portion M7, the retaining portion 27 can be accurately positioned at the first end of the second rotation path.

Exemplary illustration of steps of replacing the input module 1 is provided below using an example in which the touchpad module is replaced with the trackball module. It should be understood that the following illustration is also applicable to a case where the trackball module is replaced with the touchpad module, except that the following steps are performed in a reverse order.

Step 1: The touchpad module 1-1 is enabled to rotate along a first rotation path-1 from an end point of the first rotation path-1 to a start point of the first rotation path-1. In this process, the retaining portion 27 rotates from the second end of the second rotation path to the first end of the second rotation path with the touchpad module 1-1; the first fixing portion 1-12 of the touchpad module 1-1 is released from the mechanical connection with the second fixing portion 22 of the operation panel 2; an upper surface of the touchpad module 1-1 is located at a position higher than an upper surface of the operation panel 2, so as to facilitate the user in pulling the touchpad module 1-1 upward to unplug it, and at this time, the first electrical connection portion 1-13 of the touchpad module 1-1 is electrically disconnected from the second electrical connection portion 23 of the operation panel 2.

Step 2: The base assembly 1-211 of the trackball module 1-2 is placed at the start point of the first rotation path-2, for example, the first electrical connection portion 1-23 of the trackball module 1-2 is pressed downward into the second electrical connection portion 23 of the retaining portion 27, where the retaining portion 27 is located at the first end of the second rotation path; and the base assembly 1-211 of the trackball module 1-2 is enabled to rotate along the first rotation path-2 from the start point of the first rotation path-2 to the end point of the first rotation path-2. In this process, the retaining portion 27 rotates from the first end of the second rotation path to the second end of the second rotation path with the touchpad module 1-1; and the fifth fixing portion 1-221 of the base assembly 1-211 is mechanically connected to the seventh fixing portion 221 of the operation panel 2.

Step 3: The trackball assembly 1-212 of the trackball module 1-2 is enabled to rotate along the first rotation path-1 from the start point of the first rotation path-1 to the end point of the first rotation path-1. At this time, the sixth fixing portion 1-222 of the trackball assembly 1-212 is mechanically connected to the eighth fixing portion 222 of the operation panel 2. As such, the operation of replacing the touchpad module 1-1 with the trackball module 1-2 is completed.

It is worth noting that only the individual components or modules relevant to this embodiment have been described above, but the present application is not limited thereto. The medical input apparatus 100 may further include other components or modules. For the specifics of these components or modules, reference may be made to the related art.

According to the above embodiment, in the medical input apparatus 100, the input module 1 may be detachably connected to the operation panel 2 through a rotation movement, and by enabling the input module 1 to move along the rotation path, when the input module 1 reaches a corresponding position on the rotation path, the input module 1 may be connected to the operation panel 2 not only mechanically but also electrically, making the operation simple and reliable.

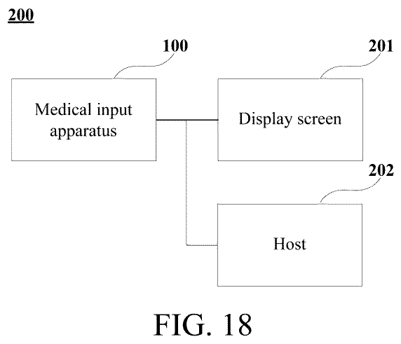

The embodiments of the present application further provide an ultrasonic imaging apparatus. FIG. 18 is a schematic diagram of an ultrasonic imaging apparatus according to an embodiment of the present application. As shown in FIG. 18, the ultrasonic imaging apparatus 200 includes the medical input apparatus 100 as described above.

As shown in FIG. 18, the ultrasonic imaging apparatus 200 may further include a display screen 201 electrically connected to the input apparatus 100 and a host 202. The host 202 can execute a control instruction received by the input apparatus 100, and an execution result of the host 202 and the like can be displayed through the display screen 201.

According to the above embodiment, in the medical input apparatus 100, the input module 1 may be detachably connected to the operation panel 2 through a rotation movement, and by enabling the input module 1 to move along the rotation path, when the input module 1 reaches a corresponding position on the rotation path, the input module 1 may be connected to the operation panel 2 not only mechanically but also electrically, making the operation simple and reliable. By providing the above-described medical input apparatus 100 in the ultrasonic imaging apparatus 200, the user experience of the user in using the ultrasonic imaging apparatus 200 can be improved.

The above embodiments merely provide illustrative descriptions of the embodiments of the present application. However, the present application is not limited thereto, and appropriate variations may be made on the basis of the above embodiments. For example, each of the above embodiments may be used independently, or one or more among the above embodiments may be combined.

The present application is described above with reference to specific embodiments. However, it should be clear to those skilled in the art that the foregoing description is merely illustrative and is not intended to limit the scope of protection of the present application. Various variations and modifications may be made by those skilled in the art according to the spirit and principle of the present application, and these variations and modifications also fall within the scope of the present application.

Preferred embodiments of the present application are described above with reference to the accompanying drawings. Many features and advantages of the implementations are clear according to the detailed description, and therefore the appended claims are intended to cover all these features and advantages that fall within the true spirit and scope of these implementations. In addition, as many modifications and changes could be easily conceived of by those skilled in the art, the embodiments of the present application are not limited to the illustrated and described precise structures and operations, but can encompass all appropriate modifications, changes, and equivalents that fall within the scope of the implementations.

1. A user input device for a medical system, comprising:
an input module comprising a body portion, a first fixing portion, and a first electrical connection portion; and
an operation panel comprising an accommodation portion for accommodating the body portion, a second fixing portion, and a second electrical connection portion,
wherein the input module rotates relative to the operation panel along a first rotation path and is detachably connected to the operation panel, the first fixing portion is connected to the second fixing portion when the input module is located at a first position on the first rotation path, and the first electrical connection portion is connected to the second electrical connection portion when the input module is located at a second position on the first rotation path.

2. The apparatus according to claim 1, wherein
the first position and the second position on the first rotation path refer to an end point of the first rotation path.

3. The apparatus according to claim 2, wherein
the first electrical connection portion is connected to the second electrical connection portion in a non-pluggable manner, and when the input module is located at the end point of the first rotation path, the first electrical connection portion and the second electrical connection portion abut against each other.

4. The apparatus according to claim 3, wherein
the first electrical connection portion and the second electrical connection portion are spring-loaded pin interfaces.

5. The apparatus according to claim 1, wherein
the first position on the first rotation path is an end point of the first rotation path, and the second position on the first rotation path is a start point of the first rotation path.

6. The apparatus according to claim 5, wherein the operation panel further comprises:
a retaining portion that is fixedly connected to the second electrical connection portion and that rotates along a second rotation path parallel to the first rotation path.

7. The apparatus according to claim 6, wherein
in a state where the first electrical connection portion is connected to the second electrical connection portion, the retaining portion rotates in synchronization with the input module.

8. The apparatus according to claim 6, wherein
the first electrical connection portion is connected to the second electrical connection portion in a pluggable manner, and when the input module is located at the start point of the first rotation path and the retaining portion is located at a first end of the second rotation path, the first electrical connection portion and the second electrical connection portion overlap each other.

9. The apparatus according to claim 6, wherein the operation panel further comprises:
a support portion comprising a first guide member;
the retaining portion comprises a second guide member, and in a state where the first guide member is engaged with the second guide, the retaining portion rotates along the second rotation path.

10. The apparatus according to claim 9, wherein
the support portion further comprises a third fixing portion;
the retaining portion further comprises a fourth fixing portion, and when the retaining portion is located at a first end of the second rotation path, the third fixing portion is connected to the fourth fixing portion.

11. The apparatus according to claim 1, wherein
the input module performs a rotational downward movement or a rotational upward movement along at least part of the first rotation path.

12. The apparatus according to claim 1, wherein
based on the input module being located at a start point of the first rotation path, an upper end face of the input module is located at a position higher than an upper end face of the operation panel; and/or
based on the input module being located at an end point of the first rotation path, the upper end face of the input module is flush with the upper end face of the operation panel.

13. The apparatus according to claim 1, wherein
the input module comprises one of a trackball module and a touchpad module; or
the input module comprises a trackball module and a touchpad module, the trackball module and the touchpad module being alternately connected to the operation panel.

14. The apparatus according to claim 13, wherein
a body portion of the trackball module comprises a base assembly and a trackball assembly, the base assembly being detachably assembled with the trackball assembly.

15. The apparatus according to claim 14, wherein
the trackball assembly comprises a ball and a cover plate, the cover plate comprises a first surface and a second surface, an opening portion passing through the first surface and the second surface is provided at the center of the cover plate, and a claw portion is provided at a side of the second surface;
part of the ball is exposed from the opening portion, and the ball is retained by the claw portion in a freely rotatable state.

16. The apparatus according to claim 14, wherein
the base assembly and the trackball assembly are each detachably connected to the operation panel;
the first fixing portion comprises a fifth fixing portion provided on the base assembly and a sixth fixing portion provided on the trackball assembly;
the second fixing portion comprises a seventh fixing portion and an eighth fixing portion that are provided in the accommodation portion;
in a state where the trackball module is mounted to the operation panel, the fifth fixing portion is connected to the seventh fixing portion, and the sixth fixing portion is connected to the eighth fixing portion.

17. The apparatus according to claim 16, further comprising a plurality of first fixing portions and a plurality of second fixing portions, and the plurality of first fixing portions and the plurality of second fixing portions are provided non-uniformly in a circumferential direction of the body portion.

18. The apparatus according to claim 1, wherein the operation panel further comprises:
a step portion extending in a circumferential direction and a position-limiting portion, the position-limiting portion and the step portion being located at upper and lower sides of the second fixing portion, respectively;
in a state where the input module is mounted to the operation panel, the first fixing portion is connected to the second fixing portion, and the first fixing portion abuts against the position-limiting portion and the step portion in an up-down direction.
